(19) United States
(12) Patent Application Publication
(10) Pub. No.: US 2004/0131692 A1
(43) Pub. Date: Jul. 8, 2004

(54) Nanoparticles made of protein with coupled apolipoprotein e for penetration of the blood-brain barrier and methods for the production thereof

(76) Inventors: Joerg Kreuter, Bad Homburg (DE); Klaus Langer, Schoneck (DE); Carolin Weber, Regensburg (DE); Renad N. Alyautdin, Moscow (RU)

Correspondence Address: BIRCH STEWART KOLASCH & BIRCH, PO BOX 747, FALLS CHURCH, VA, 22040-0747, US

(21) Appl. No.: 10/476,653

(22) PCT Filed: Apr. 30, 2002
(86) PCT No.: PCT/EP02/04735

(30) Foreign Application Priority Data
May 5, 2001 (DE) 101 21 982.2

(51) Int. Cl. A61K 9/50

(52) U.S. Cl. 424/499

(57) ABSTRACT

The present invention relates to nanoparticles for penetrating the blood-brain barrier. The nanoparticles are characterized in that they consist of a hydrophile protein or of a combination of hydrophile proteins, preferably of serum albumin, with particular preference of human origin, to which apolipoprotein E is coupled. The invention further relates to processes for the manufacture of such nanoparticles.

The present invention relates to Nanoparticles made of a hydrophile protein or of a combination of hydrophile proteins, preferably of serum albumin, especially of human origin, which by means of bound apolipoprotein E are able to cross the blood-brain barrier in order to transport pharmaceutically or biologically active agents into the liquor cerebrospinalis.

Nanoparticles are particles having a size between 10 and 1000 nm which can be manufactured from artificial or natural macromolecular substances. To such nanoparticles can be bound drugs or other biologically active materials by covalent, ionic or adsorptive linkage, or the latter can be incorporated into the material of the nanoparticles.

To this day, however, only nanoparticles of polyalcylcyanoacrylates which were coated with polysorbate 80 (Tween® 80) or other tensides were capable of crossing the blood-brain barrier in order to transport hydrophile drugs to the liquor cerebrospinalis and induce pharmacological effects. According to existing studies, the mechanism of this transport is based on apolipoprotein E (ApoE) being adsorbed by the nanoparticles via the polysorbate 80 coating. Presumably, these particles thereby pretend to be lipoprotein particles, which are recognized and bound by the LDL receptors of the cerebrocapillary endothelial cells which ensure the lipid supply to the brain.

It was possible to transport a number of drugs by means of polybutylcyonoacrylate nanoparticles coated with polysorbate 80 or other tensides across the blood-brain barrier and cause a significant pharmacological effect. Examples for drugs administered in this way are dalargin, an endorphin hexapeptide, loperamide and tubocuarine, the two NMDA receptor antagonists MRZ 2/576, respectively MRZ 2/596 (Merz, Frankfurt), as well as the anticancer drug doxorubicin.

The disadvantages of the polybutylcyanoacrylate nanoparticles are that polysorbate 80 is not physiological and that the transport across the blood-brain barrier may possibly be due to a toxic effect of polysorbate 80. A coating of polybutylcyanoacrylate nanoparticles with polysorbate 80 or other tensides is, however, essential for the transport of the polybutylcyanoacrylate nanoparticles across the blood-brain barrier. However, the known polybutylcyanoacrylate nanoparticles have the further disadvantage that both the binding of the ApoE as well as that of the drugs takes place only by adsorption. Thereby, the nanoparticle-bound ApoE or drug is present in equilibrium with free APOE, respectively with free drug, and, after injection into the body, rapid desorption of these substances from the particles may occur. In addition, most drugs do not bind to a sufficient extent to polybutylcyanoacrylate nanoparticles and can therefore not be transported across the blood-brain barrier with the aid of this carrier system.

The task underlying the present invention was to provide nanoparticles for passing through the blood-brain barrier which do not have the aforementioned disadvantages and which, under avoidance of non-physiological tensides, do not have the apolipoprotein E, which is necessary for the transport across the blood-brain barrier, merely adsorbed thereto.

The task was surprisingly solved by nanoparticles consisting of a hydrophile protein or a combination of hydrophile proteins, preferably of serum albumin, with particular preference of human serum albumin, or of a comparable protein, to which lipoprotein E is coupled covalently or via the avidin/biotin system.

Albumins are a group of proteins occurring above all in animal/human liquids, e.g. the serum albumin in the blood, or tissues. Albumins are rich in negatively charged amino acids as well as leucine and isoleucine. Compared to the globulins accompanying the albumins, albumins have a lower molecular mass and are precipitable only by relatively high salt concentrations.

Gelatine A, gelatine B, casein or comparable proteins are also suitable as starting proteins for the inventive nanoparticles.

Apolipoprotein E is a component of the lipoprotein complexes. These complexes of lipids and apolipoproteins enable the transport of the lipids, which are insoluble in water, in the blood. ApoE presumably mediates the transport of the inventive nanoparticles across the blood-brain barrier by binding to the LDL receptors of the cerebrocapillary endothelial cells.

The inventive nanoparticles may additionally have one or more functional proteins bound via bifunctional spacer molecules to thiol groups of the thiol group-modified nanoparticles. To prepare such nanoparticles it is possible for the functional groups located on the surface of the nanoparticles (amino groups, carboxyl groups, hydroxyl groups) to be converted by suitable reagents to reactive thiol groups. Functional proteins can then be bound to the thiol group-modified nanoparticles via bifunctional spacer molecules having reactivity both to amino groups as well as to free thiol groups.

The functional proteins to be coupled in this way to the nanoparticles may be selected from the group comprising avidin, avidin derivatives, apolipoproteins such as apolipoprotein E, but also antibodies, enzymes and the like. In this context, the functional proteins themselves may have pharmacological or biological action.

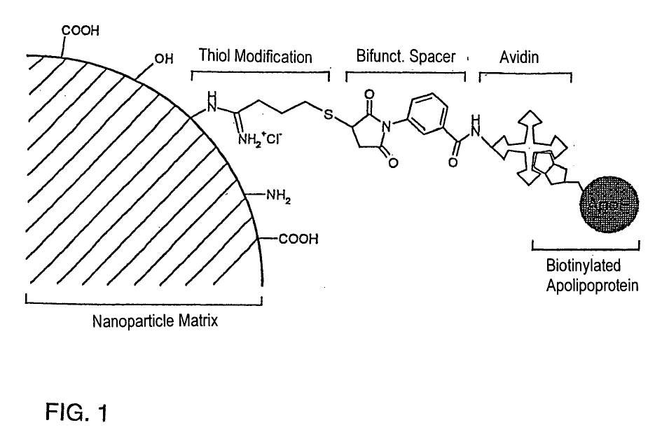

In a preferred embodiment, the inventive nanoparticles have covalently coupled avidin, via which biotinylated apolipoprotein E can be bound as is illustrated in FIG. 1. Avidin itself is a glycoprotein which is highly affine to biotin and is covalently bound via the aforementioned bifunctional spacer molecules to the thiol groups of the thiolated nanoparticles. By the covalent linkage of the avidin to the nanoparticles it is not only possible to bind biotinylated ApoE, which is necessary for the transport to the blood-brain barrier, but also to bind a variety of biotinylated molecules to the avidin-modified nanoparticles in a particularly efficient manner. For this purpose, pharmacologically or biologically active molecules are especially preferred.

To impart pharmacologic effects, the inventive nanoparticles may have pharmacologically or biologically active substances. These active substances may be incorporated in the nanoparticles, or they are bound by the nanoparticles. The binding of the pharmacologically or biologically active agents may be performed covalently, with complex-formation via the avidin-biotin system, as well as incorporatively or adsorptively.

The inventive nanoparticles are particularly suitable for binding drugs which have no passage or an insufficient passage across the blood-brain barrier, for instance dalargin, loperamid, tubocuarin or doxorubicin or the like, and for transporting them across the blood-brain barrier and inducing pharmacological effects.

The method of preparing the inventive nanoparticles of a hydrophile protein or a combination of hydrophile proteins with the purpose of crossing the blood-brain barrier comprises the following steps:

desolvating an aqueous solution of a hydrophile protein or a combination of hydrophile proteins,

stabilising the nanoparticles produced by the desolvation by crosslinking,

converting a part of the functional groups on the surface of the stabilised nanoparticles to reactive thiol groups,

covalently attaching functional proteins by means of bifunctional spacer molecules,

biotinylating apolipoprotein E if the particles do not have covalently coupled apolipoprotein E,

loading the avidin-modified nanoparticles with biotinylated ApoE and with the pharmacologically active agent to be administered.

To prepare the nanoparticles a hydrophile protein or a combination of hydrophile proteins is used as starting material. With preference, an aqueous solution of serum albumin, with particular preference of human serum albumin, is desolvated under stirring. The nanoparticles being formed are stabilised by crosslinking, and the functional groups (amino groups, carboxyl groups, hydroxyl groups) on the surface of the nanoparticles are converted by suitable reagents to reactive thiol groups.

Desolvation from the aqueous solvent is preferably performed by addition of ethanol. In principle, desolvation is also possible by adding other water-miscible non-solvents for hydrophile proteins such as acetone, isopropanol or methanol. Thus, gelatine as starting protein was successfully desolvated by addition of acetone. At the same time it is possible to perform desolvation of proteins dissolved in aqueous phase by adding structure-forming salts such as magnesium sulphate or ammonium sulphate. This is called salting-out.

Suitable as cross-linker for stabilising the nanoparticles are bifunctional aldehydes, preferably glutaraldhehyde, as well as formaldehyde. Furthermore, crosslinking of the nanoparticle matrix is possible by thermal processes. Stable nanoparticle systems were obtained at 60° C. over periods of more than 25 hours, or 70° C. over periods of more than 2 hours.

Thiolation of the nanoparticle surface can be performed according to various principles. By preference, the amino groups on the particle surface are converted with 2-iminothiolane, which reacts with primary amino groups on the particle surface, to free thiol groups on the particle surface. Apart from this, thiol groups can also be obtained by reductive cleavage of disulfide bonds with dithiotreitol (DTT), which disulfide bonds are present on the surface of the nanoparticle matrix. As an alternative, free carboxyl groups of the particle surface can be converted with 1-ethyl-3-(3-dimethylaminopropyl)carbodiimide (EDC)/cysteine, or with EDC/cystaminium dichloride, and the disulfide bonds thus introduced can subsequently be reductively cleaved with DTT.

Functional proteins can be coupled to the thiol groups-modified nanoparticles via bifunctional spacer molecules which have reactivity both to amino groups as well as to free thiol groups. Applicable are heterobifunctional spacer molecules with reactivities to carboxyl or hydroxyl groups but also homobifunctional spacer molecules with reactivities to amino groups. A preferred substance able to take over the function of a bifunctional spacer molecule is m-maleimidobenzoyl-N-hydroxysulfosuccinimide ester (sulfo-MBS). Apart from m-maleimidobenzoyl-N-hydroxysulfosuccinimide ester, further heterobifunctional spacer molecules have also been successfully utilized, such as sulfosuccinimidyl-4-[N-maleimidomethyl]-cyclohexane-1-carboxylate (sulfo-SMCC) or sulfosuccinimidyl-2-[m-azido-o-nitrobenzamido]-ethyl-1,3′-dithiopropionate (SAND), as well as the homobifunctional spacer molecules dimethyl-3,3′-dithiobispropionimidate-dihydrochloride (DTBP) or 3,3′-dithiobis[sulfo-succinimidylpropionate] (DTSSP). Heterobifunctional spacer molecules are, however, preferred since homobifunctional spacer molecules also lead to a possible intramolecular crosslinking as a side reaction to the attachment of functional proteins to the nanoparticle surface.

In a particularly preferred method, avidin or an avidin derivative is coupled to the thiolated nanoparticles by the bifunctional spacer molecules. This intermediate product, avidin-modified nanoparticles, represents a universal carrier system for a variety of biotinylated substances which can be bound via avidin-biotin complex formation.

To bind the human apolipoprotein E to the avidin-modified nanoparticles, the apolipoprotein E can be biotinylated by conversion with N-hydroxysuccinimidobiotin (NHS biotin). Other biotinylating reagents reacting with amino groups or other functional groups of the protein to be bound can also be utilized. For biotinylation, free sulfhydryl groups or carboxyl groups, as further functional groups of the protein to be bound, are also suitable. Alternative biotinylating reagents for amino groups differ from the NHS biotin in their aminoreactive functionality, for instance by possessing pentafluorophenyl groups instead of succinimido groups, or in the region between biotin and the aminoreactive functionality.

To induce pharmacological effects, pharmaceutically or biologically active substances are incorporated in the particles, or directly or indirectly bound to the avidin-modified nanoparticles. The avidin-modified nanoparticles can be loaded, simultaneously or in any order desired, with biotinylated apolipoprotein E and a pharmaceutically active agent. The binding of the active agent may be performed by covalent linkage, complexing linkage via the avidin-biotin system, as well as by adsorptive linkage.

The inventive nanoparticles of a hydrophile protein or a combination of hydrophile proteins having apolipoprotein E bound thereto are suitable for transporting pharmaceutically or biologically active agents which otherwise would not be able to pass through the blood-brain barrier, in particular hydrophile active agents, across the blood-brain barrier and to induce pharmacological effects. Examples of such active agents are dalargin, loperamide, tubocuarine, doxorubicin and the like.

Thus, the active agent-loaded nanoparticles are suitable for treatment of a large number of cerebral diseases. The active agents bound to the carrier system are selected according to the respective therapeutic aim. The carrier system suggests itself above all for those active substances which show no passage or an insufficient passage across the blood-brain barrier. Considered as active substances are zytostatic agents for the therapy of cerebral tumours, active substances for the therapy of viral infections in the cerebral region e.g. HIV infections, but also active substances for the therapy of dementia affections, to mention but a few application areas.

FIG. 1 shows a preferred embodiment of the inventive nanoparticles, without active substance or a further functional protein.

In the following, the invention will be illustrated with reference to an example of an embodiment. This representation is not to be understood as in any way limiting the meaning and spirit of the present invention.

To prepare nanoparticles from human serum albumin (HSA), 200 mg of human serum albumin was dissolved in 2.0 ml of purified water. To this solution was added 8.0 ml of 96%-vol ethanol by drop-wise addition under stirring with a magnetic stirrer (500 rpm).

The resultant nanoparticles were stabilized by adding to the reaction mixture 235 μl of an aqueous, 8% (m/v) glutaraldehyde solution, and stirring for 24 hours at room temperature. The stabilized nanoparticles were purified by centrifuging five times (16,000 rcf, 8 min) and redispersing in 1.5 ml of purified water. The resultant content of nanoparticles in the suspension was determined by gravimetric determination.

Subsequently, 2.0 ml of a solution of 13 mg 2-iminothiolane (Traut's reagent) in tris-buffer (pH 8.5) were added to 2.0 ml of nanoparticle suspension and stirring was performed for 24 hours to thiolate the particle surface. After thiolating, the nanoparticles were purified as described above.

The avidin derivative NeutrAvidin™ was covalently bound to the thiolated nanoparticles via m-maleimidobenzoyl-N-hydroxysulfosuccinimide ester (sulfo-MBS), a substance functioning as bifunctional spacer molecule. To this end, the avidin derivative was activated by adding 1.6 mg of sulfo-MBS to a solution of 5.0 mg of NeutrAviding™ in 1.0 ml PBS buffer (pH 7.0) and stirring was performed for 1 hour at room temperature. Free sulfo-MBS was separated from the activated NeutrAvidin by size exclusion chromatography.

The fractions in which, by sprectrophometric detection at 280 nm wavelength, NeutrAvidin could be detected were united and 2.0 ml of the thiolated nanoparticles were added thereto, and stirring was performed for 1 hour at room temperature. The avidin-modified HSA nanoparticles were purified as described above.

Apolipoprotein E (ApoE) was biotinylated by dissolving 250 μg of ApoE in 125 μl of isotonic PBS buffer, pH 7.4, and by adding a solution of 150 μg of NHS biotin (N-hydroxysuccinimidobiotin) in 15 μl of DMSO to the said solution. After a reaction time of 2 hours at 10° C. under stirring, this mixture was diluted with a further 300 μl of PBS buffer, pH 7.4. Still free NHS biotin was separated from the biotinylated ApoE by size exclusion chromatography. The fractions wherein, by photometric detection at a wavelength of 280 nm, ApoE could be detected were purified and freeze-dried.

The avidin-modified HSA nanoparticles were loaded, immediately prior to the animal experiment, with the biotinylated ApoE and with the drug dalargin. To this end, the freeze-dried ApoE was dissolved in 250 μl of distilled water and added thereto was 280 μl of an HSA nanoparticle suspension containing 5.9 mg of avidin-modified HSA nanoparticles. A solution of 1.125 mg of dalargin in 470 μl of water was added, and the mixture was incubated for 3 hours at room temperature. After this incubation, the mixture was diluted by adding 500 μl of isotonic PBS buffer, pH 7.4.

A quantification of the loading of avidin-modified HSA nanoparticles with dalargin showed that given a ratio of dalargin/nanoparticles=191 μg/mg, adsorptive binding of 23.7 μg/mg (=12.4%) of dalargin occurred.

The ready-to-be-applied preparation contained, in a total volume of 1.5 ml of isotonic PBS buffer:

3.93 mg/ml of avidin-modified HSA nanoparticles

167 μg/ml of ApoE (bound to the nanoparticles via the avidin-biotin system)

0.75 mg/ml of dalargin (12.4% of which bound adsorptively to nanoparticles).

The preparation was applied to mice in vitro at a dosage of 7.5 mg/kg of dalargin. This corresponds to an application amount of 200 μl of the aforementioned preparation per mouse, based on an average body weight of a mouse of 20 g.

The analgesic effect (nociceptive response) was determined by the tail-flick test, in which a hot beam of light is projected onto the tail of the mouse and the time until the mouse draws away its tail is measured. After 10 seconds (=100% MPE) the experiment was truncated so as not to cause injury to the mouse. The maximally possible analgesic effect (MPE) was determined in accordance with the following formula:

$$\% MPE = \frac{\begin{matrix}response time after application - \\ response time prior to application\end{matrix}}{cut \text{-} off time - response time prior to application} \times 100$$

Negative MPE values result if after administration of the administration the mouse draws away its tail before the treatment.

With the aid of dalargin-loaded avidin-modified HSA nanoparticles the analgesic effects indicated in Table 1 were obtained after intravenous injection.

TABLE 1
Analgesic effect [% MPE] in mice (n = 6)
after i.v. application of dalargin (7.5 mg/kg)
in the form of one of the preparations indicated.
(MPE = Maximal Possible Effect)

| Preparation | 30 min | 45 min | 90 min | 120 min |
|---|---|---|---|---|
| HSA-avidin- | 25.1 ± 12.4 | 49.0 ± 23.7 | 2.1 ± 19.6 | −0.23 ± 12.3 |
| nanoparticles + | | | | |
| ApoE + dalargin | | | | |
| Controls* | | | | |
| HSA-avidin- | −2.6 ± 3.9 | −5.4 ± 10.9 | −14.4 ± 17.4 | −9.6 ± 20.6 |
| nanoparticles + dalargin | | | | |
| PBCA- | 35.2 ± 5.8 | 49.5 ± 4.5 | 36.5 ± 13.7 | 7.1 ± 6.3 |
| nanoparticles + | | | | |
| dalargin + | | | | |
| polysorbate 80 | | | | |
| Dalargin solution | 10.0 ± 9.8 | 9.3 ± 2.8 | 4.7 ± 5.1 | 2.0 ± 6.1 |

*The PBCA nanoparticles + dalargin + polysorbate 80 and the dalargin solution comparison data originate from an earlier experiment

The results show that with the avidin-modified HSA nanoparticles it was possible to achieve analgesic effects that correspond to the effects achieved with the polybutylcyanoacrylate nanoparticles (PBCA nanoparticles).

1. Nanoparticles for crossing the blood-brain barrier, characterized in that they consist of a hydrophile protein or of a combination of hydrophile proteins to which apolipoprotein E is coupled or bound.

2. Nanoparticles according to claim 1, characterized in that at least one hydrophile protein is selected from the group comprising serum albumin, gelatine A, gelatine B, casein and comparable proteins, or comprises a combination of these proteins.

3. Nanoparticles according to claim 1 or 2, characterized in that at least one hydrophile protein is of human origin.

4. Nanoparticles according to any one of the preceding claims, characterized in that they have one or more different functional proteins which are bound via bifunctional spacer molecules to thiol groups of thiol group-modified nanoparticles.

5. Nanoparticles according to claim 4, characterized in that the functional proteins are selected from the group comprising avidin, avidin derivatives, apolipoproteins, antibodies, enzymes, hormones, zytostatic agents and the like.

6. Nanoparticles according to claim 5 characterized in that the biotinylated apolipoprotein E is bound via covalently coupled avidin.

7. Nanoparticles according to claim 6, characterized in that at least one further biotinylated functional protein is bound via covalently coupled avidin.

8. Nanoparticles according to any one of the preceding claims characterized in that they have incorporated therein or bound thereto pharmacologically or biologically active agents.

9. Nanoparticles according to claim 8, characterized in that the pharmacologically or biologically active agents are bound on the particle surface.

10. Nanoparticles according to claim 8, characterized in that the pharmacologically or biologically active agents are bound covalently, or by complex formation via the avidin-biotin system, or adsorptively.

11. Nanoparticles according to any one of claims 8 to 10, characterized in that the active agents are selected from the group comprising dalargin, loperamide, tubocuarine and doxorubicin.

12. Process for the manufacture of nanoparticles of one hydrophile protein or a combination of hydrophile proteins for crossing the blood-brain barrier, characterized in that it comprises the following steps:
desolvating an aqueous solution of a hydrophile protein or a combination of hydrophile proteins,
stabilising the nanoparticles produced by the desolvation by crosslinking,
converting a part of the functional groups on the surface of the stabilised nanoparticles to reactive thiol groups,
covalently attaching functional proteins, preferably avidin, by means of bifunctional spacer molecules,
biotinylating the apolipoprotein E,
loading the avidin-modified nanoparticles with biotinylated apolipoprotein ApoE
loading the avidin-modified nanoparticles with biotinylated apolipoprotein E and with further functional proteins or pharmaceutically or biologically active substances.

13. Process according to claim 12, characterized in that the hydrophile protein is selected from the group comprising serum albumin, gelatine A, gelatine B, casein and comparable proteins, or comprises a combination of these proteins.

14. Process according to claim 12 or 13, characterized in that the hydrophile protein is of human origin.

15. Process according to claims 12 to 14, characterized in that the desolvation is performed by stirring and adding a water-miscible non-solvent for hydrophile proteins or by salting-out.

16. Process according to claim 15, characterized in that the water-miscible non-solvent for hydrophile proteins is selected from the group comprising ethanol, methanol, isopropanol and acetone.

17. Process according to any one of claims 12 to 16, characterized in that for stabilizing the nanoparticles, thermal processes or bifunctional aldehydes or formaldehyde is used.

18. Process according to claim 17, characterized in that glutaraldehyde is used as bifunctional aldehyde.

19. Process according to any one of claims 12 to 18, characterized in that as thiol group-modified agent a substance is used which is selected from the group comprising 2-iminothiolane, a combination of 1-ethyl-3-(3-dimethylaminopropyl)carbodiimide and cysteine, or a combination of 1-ethyl-3-(3-dimethylaminopropyl)carbodiimide and cystaminium dichloride as well as dithiotreitol.

20. Process according to one of claims 12 to 19, characterized in that as bifunctional spacer molecule a substance is used which is selected from the group comprising m-maleimidobenzoyl-N-hydroxysulfosuccinimide ester, sulfosuccinimidyl-4-[N-maleimidomethyl]-cyclohexane-1-carboxylate, sulfosuccinimidyl-2-[m-azido-o-nitrobenzamido]-ethyl-1,3′-dithiopropionate, dimethyl-3,3′-dithiobispropionimidate-dihydrochloride and 3,3′-dithiobis[sulfosuccinimidylpropionate].

21. Process according to any one of claims 12 to 20, characterized in that the active substances are selected from the group comprising dalargin, loperamide, tubocuarine and doxorubicin.

22. Use of nanoparticles which comprise a hydrophile protein or a combination of hydrophile proteins which have bound thereto apolipoprotein E, for transport of pharmaceutically or biologically active agents across the blood-brain barrier.

23. Use according to claim 22, characterized in that at least one of the hydrophile proteins is selected from the group comprising serum albumin, gelatine A, gelatine B, casein and comparable proteins, or comprises a combination of these proteins.

24. Use according to claim 22 or 23, characterized in that at least one of the hydrophile proteins is of human origin.

25. Use of nanoparticles according to any one of claims 22 to 24, characterized in that the active agents are selected from the group comprising dalargin, loperamide, tubocuarine and doxorubicin.

26. Use of nanoparticles according to any one of claims 22 to 25, characterized in that it is used for treating cerebral affections.
